(19) United States
(12) Patent Application Publication
(10) Pub. No.: US 2007/0225678 A1
(43) Pub. Date: Sep. 27, 2007

(54) Two-way valved catheter

(75) Inventor: Chun Kee Lui, Monroeville, PA (US)

Correspondence Address: BRINKS HOFER GILSON & LIONE/INDY/COOK, ONE INDIANA SQUARE, SUITE 1600, INDIANAPOLIS, IN, 46204-2033, US

(73) Assignee: Cook Vascular Incorporated, Leechburg, PA (US)

(21) Appl. No.: 11/725,748

(22) Filed: Mar. 20, 2007

Related U.S. Application Data
(60) Provisional application No. 60/785,196, filed on Mar. 23, 2006.

(51) Int. Cl. A61M 25/00; A61M 25/16; A61M 25/18

(52) U.S. Cl. 604/523; 604/537

(57) ABSTRACT

A catheter and method for transporting a fluid between a body vessel of a patient and a site external of the patient. The catheter comprises a catheter body sized to at least partially extend between the body vessel and the external site. The catheter body has a proximal portion extending toward the external site and a distal portion extending to the body vessel. An inwardly contoured segment is formed in the catheter body at the distal portion, and a valve is formed at the inwardly contoured segment. The valve is operable to open in a first direction when subjected to a first pressure from a side of the valve to permit fluid flow therethrough in the first direction, and in a second direction opposite the first direction when subjected to a second pressure from another side of the valve to permit fluid flow therethrough in the second direction.

### RELATED APPLICATION

The present patent document claims the benefit of the filing date under 35 U.S.C. § 119(e) of Provisional U.S. Patent Application Ser. No. 60/785,196, filed Mar. 23, 2006, which is hereby incorporated by reference.

### BACKGROUND

1. Technical Field

This invention relates generally to a catheter having a valve for controlling the flow of a fluid therethrough, and more particularly, to a catheter fabricated from a performance material having a two-way valve formed therein.

2. Background Information

Valved catheters are well known in the medical arts. Typically, a valve is positioned in a lumen of a catheter to selectively control, or prevent, the unidirectional flow of fluid through the lumen. Some catheters are provided with two-way valves. Two-way valves are structured to permit fluid to flow therethrough in either direction upon occurrence of a designated condition, typically upon the occurrence of a predetermined pressure differential between the respective sides of the valve.

One such two-way valve is described in U.S. Pat. No. 4,549,879 to Groshong. The catheter in the '879 patent is formed of a soft, flexible material (silicone rubber), and has a slit valve formed in a catheter wall. The slit valve is closed under normal physiologic pressures. In this event, the catheter walls on either side of the slit are in registry with each other, and fluid cannot pass through the valve. When sufficient pressure gradients are applied across the slit valve, the catheter walls deform such that the slit valve surfaces are no longer in registry, and an orifice is formed through which fluid may flow into, or out of, the catheter. As shown in FIG. 4b of the '879 patent, the valve opens outwardly when the fluid pressure inside the catheter exceeds the fluid pressure outside the catheter by a predetermined amount. As a result, a pressurized fluid within the catheter infuses through the valve to the region exterior of the catheter. Similarly, as shown in FIG. 4c, when the pressure differential outside the catheter exceeds that interiorly of the catheter by a predetermined amount, the valve opens inwardly to permit aspiration of a fluid, such as blood, into the interior of the catheter.

There are some disadvantages associated with the use of two-way valves in soft, flexible catheters. For example, since the catheter of the '879 patent is formed of a soft material, the catheter cannot be easily navigated through the vasculature. In order to generate sufficient force to push the catheter through a vessel, a stiffening wire must be inserted into the lumen of the catheter in a manner such that it abuts the closed end of the catheter. A force is exerted against the wire, and thus against the closed catheter end, to direct the catheter along the desired pathway. Another disadvantage associated with the use of valves formed from such soft materials, is that such valves are subject to unintended opening generated by unexpected and/or involuntary physical phenomena, such as coughing, on the part of the patient.

Higher performance catheter materials have sufficient strength to overcome these disadvantages; however such performance materials are often too stiff to allow reliable operation of a two-way valve. For example, the pressure differentials required to open such valves may be undesirably high. In addition, the valves may lack the flexibility to reliably return to their original sealed condition following cessation of the pressure. Still further, the lack of flexibility of such valves renders them subject to leakage. Even if such catheters can be structured to operate as a valve, such valves may only be reliable for re-sealing after fluid flow in a first direction, and are not operable as two-way valves.

Long-term implantable catheters have traditionally been fabricated from silicone (a thermoset material), or thermoplastic polyether-based polyurethane. The Groshong catheter described above is an example of a catheter formed from silicone. The Groshong catheter was fabricated from a soft silicone because that composition was necessary for that valve to work, i.e., the catheter was designed for the valve. Polyether-based polyurethane catheters typically exhibit some properties superior to those of silicone, e.g., higher tensile strength and fatigue resistance. However, polyether-based polyurethane is subject to stress cracking when used in long-term implants in the human body.

It is desired to provide a catheter having a two-way valve formed therein, in which the catheter is formed of a material having a high degree of strength and pushability, and does not exhibit appreciable stress-cracking upon long-term implantation. It is further desired to provide a catheter formed from a material wherein the two-way valve is structured such that it opens upon exposure to a desired pressure differential that may be generated from either side of the valve, and reliably re-seals upon release or diminution of the pressure.

### SUMMARY

The present invention addresses the problems existing in the art. In one form thereof, the invention comprises a catheter for use in transporting a fluid between a body vessel of a patient and a site external of the patient. The catheter comprises a catheter body sized to at least partially extend between the body vessel and the external site. The catheter body has a proximal portion extending toward the external site and a distal portion extending to the body vessel. An inwardly contoured segment is formed in the catheter body at the distal portion, and a valve is formed at the inwardly contoured segment. The valve is operable to open in a first direction when subjected to a first pressure from a side of the valve to permit fluid flow therethrough in the first direction, and in a second direction opposite the first direction when subjected to a second pressure from another side of the valve to permit fluid flow therethrough in the second direction.

In another form thereof, the invention comprises a method of forming a two-way valved catheter for transporting a fluid between a body vessel of a patient and a site external of the patient. A tubular catheter body sized such that a proximal portion extends substantially to the external site and a distal portion extends to the body vessel is provided, and an inwardly contoured segment is formed at the distal portion of the tubular catheter body. A valve is formed at the inwardly contoured segment, which valve comprises a longitudinal slit formed in the contoured segment. The valve is sized and dimensioned such that upon exposure to a first predetermined pressure from a first side thereof, the valve opens in a first direction to permit fluid flow therethrough in the first direction, and upon exposure to a second predetermined pressure from a second side thereof, the valve opens in a second direction, opposite to the first direction, to permit fluid flow therethrough in the second direction. The valve is structured to remain closed in the absence of the predetermined first and second pressures.

### BRIEF DESCRIPTION OF THE DRAWINGS

FIG. 1 is a side elevational view of the distal end of a catheter according to an embodiment of the present invention, showing the two-way valve in the closed position;

FIG. 2 is a sectional view taken along line 2-2 of FIG. 1;

FIG. 3 is a sectional view taken along line 3-3 of FIG. 1;

FIG. 4 is a sectional view of a mold and mandril for forming the two-way valve in the catheter, showing the catheter positioned between upper and lower mold portions;

FIG. 5 is an enlarged view of mandril 20;

FIG. 6 is an enlarged cross-sectional view of the contoured portion of the catheter body following removal from the mold;

FIG. 7 is a side elevational view of the distal end of another embodiment of a valved catheter according to an embodiment of the present invention;

FIG. 8 is a sectional view taken along line 8-8 of FIG. 7;

FIG. 9 is a sectional view of another embodiment of a valved catheter, wherein the catheter is co-extruded from a stiff and a soft material;

FIG. 10 is a side elevational view of the distal end of another embodiment of a valved catheter according to an embodiment of the present invention, wherein the catheter includes a one-way valve and a two-way valve;

FIG. 11 is a top view of the catheter of FIG. 10; and

FIG. 12 is a sectional view taken along line 12-12 of FIG. 10.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

In the following discussion, the terms “proximal” and “distal” will be used to describe the opposing axial ends of the catheter, as well as the opposing axial ends of various component features. The term “proximal” is used in its conventional sense to refer to the end of the catheter (or component thereof) that is closest to the operator during use of the device. The term “distal” is used in its conventional sense to refer to the end of the catheter (or component thereof) that is initially inserted into the patient, or that is closest to the patient.

FIG. 1 is a side elevational view of the distal end portion of a catheter 10, according to an embodiment of the present invention. Catheter 10 includes a tubular catheter body 11 having a lumen 16 formed therein, and a distal tip 14 welded or otherwise securely engaged at the distal end of tubular catheter body 11. Preferably, distal tip 14 is formed from the same or a similar composition used to form catheter body 11.

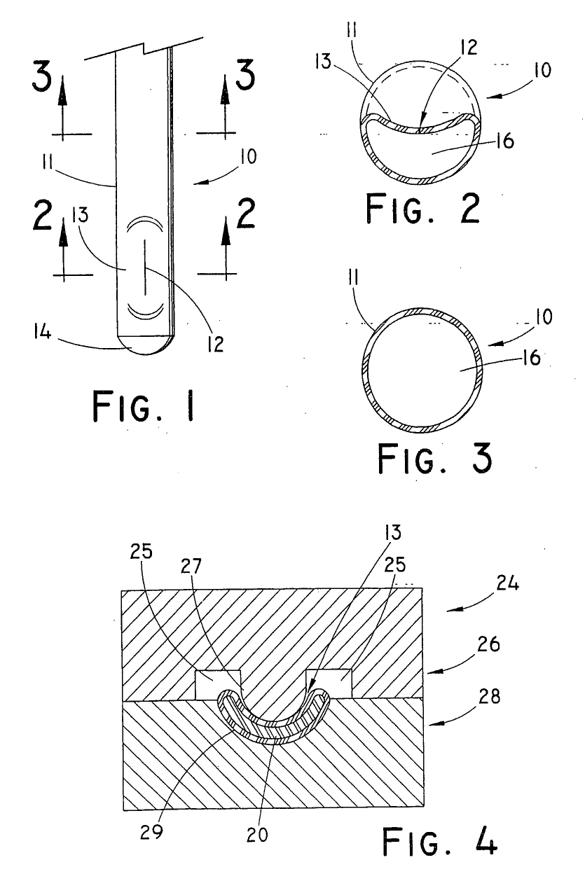

Catheter body 11 includes a valve slit 12 disposed proximally of distal tip 14. Valve slit 12 is provided in an inwardly contoured segment 13 of the distal end portion of catheter body 11. As used herein, an “inwardly contoured” segment refers to a segment of the catheter body that is inverted, depressed or otherwise sunken with respect to the remainder of the catheter body. The inwardly contoured segment is best shown in FIG. 2, taken along line 2-2 of FIG. 1. For comparison, FIG. 3, taken along line 3-3 of FIG. 1, illustrates the remainder of (non-contoured) catheter body 11 proximal of contoured segment 13 in cross section. The inwardly contoured segment extends in the proximal direction a selected distance from distal tip 14 of catheter 10, such as about one-half inch (1.3 cm). In FIGS. 1 and 2, valve slit 12 is shown in its closed position. If desired, the inwardly contoured portion may extend all the way to the distal end of catheter 10.

Unlike thermoset silicone elastomers that have been conventionally utilized to form catheters having two-way valves, the present invention preferably utilizes a thermoplastic material that is capable of exhibiting superior mechanical properties when compared to the prior materials. Thermoplastic and thermoset materials are processed quite differently. In a preferred embodiment, the material used to form the inventive catheter comprises a performance material capable of functioning in reliable fashion as a two-way valve, and that is also suitable for long-term implantation without appreciable stress-cracking. As the term is used herein, a “performance material” is a material having sufficient pushability to enable the catheter to be directed through a vessel, sufficient tensile strength to resist compressive forces typically encountered upon passage through the vessel, sufficient freedom from stress-cracking under normal conditions encountered within a vessel to enable long-term (e.g., at least 30 days or more) implantation of the catheter, and which is capable of having a two-way valve formed therein, which valve is capable of opening in a first direction in response to a desired pressure differential across the valve originating from one side of the valve, and opening in a second direction in response to a desired pressure differential across the valve originating from an opposite side of the valve, and reseals when the pressure differential is not present.

Particularly preferred performance materials comprise a class of materials known as polycarbonate urethanes. Polycarbonate urethanes are thermoplastic elastomers formed as the reaction product of a hydroxyl terminated polycarbonate, an aromatic diisocyanate, and a low molecular weight glycol used as a chain extender. The carbonate linkages adjacent to the hydrocarbon groups give this class of materials high oxidative stability, rendering them particularly beneficial in applications such as long-term implantation, wherein oxidation may otherwise lead to premature degradation. The materials also have high biocompatibility, pushability and mechanical strength. Further discussion of polycarbonate urethanes, and their preparation, is provided in, e.g., U.S. Pat. Nos. 5,133,742 and 4,810,749, incorporated by reference herein. Polycarbonate urethanes suitable for use herein are available commercially from, e.g., The Polymer Technology Group, of Berkeley, CA, under the trademark BIONATE®, and from the CT Materials division of Cardiotech International Inc., under the trademark CHRONOFLEX®. CHRONOFLEX® polycarbonate urethanes are available as an aliphatic thermoplastic polymer (CHRONOFLEX® AL), and as an aromatic thermoplastic polymer (CHRONOFLEX® AR).

In addition to the commercial products listed above, suitable polycarbonate urethanes can be custom synthesized in known manner to optimize the specific properties desired for a particular application. Although polycarbonate urethanes comprise the preferred materials for use herein, other performance materials capable of exhibiting the properties described herein may be substituted.

When an inwardly contoured catheter is formed as described hereinafter, the catheter comprises a reliably-functional two-way valve that is built into the wall of the catheter. The valved catheter has the strength, durability and freedom from stress-cracking not shown in prior art valved catheters, such as silicone valved catheters and catheters formed from other conventional non-performance materials.

As stated, the two-way valve comprises a longitudinal slit in an inwardly contoured segment of the catheter body. The slit is structured such that the valve opens when subjected to a predetermined positive or negative pressure, and remains closed when there is no pressure differential across the valve, or where a pressure differential exists, but the differential is less than a predetermined level required for opening the valve in the desired direction. The formation of one-way valves in performance catheters is relatively straightforward, and generally does not give rise to troublesome structural issues. However, providing a two-way valve in such catheters to permit fluid to flow in both directions, and to effectively re-seal following such flow, has been problematic. The present performance catheter is structured in a manner to overcome such problems by permitting such two-way flow, and effectively re-sealing following the flow. By providing the slit in an inwardly contoured segment of the catheter as described, and by structuring the walls of the catheter in a manner such that the slit is disposed at particular geometries relative to the wall, a very reliable and efficient two-way valve is formed.

In a preferred embodiment, the two-way valve may be formed in the following manner. The inwardly contoured segment of the catheter body is initially formed into a desired shape. One preferred manner in which this portion may be shaped is by the application of heat and pressure in a mold. One arrangement for accomplishing this is illustrated in FIG. 4. A suitably sized and shaped mandril 20 is inserted inside the portion 13 of catheter body 111 to be contoured, and the catheter portion is positioned in a suitably sized and shaped mold 24. Mold 24 has an upper mold portion 26 and a lower mold portion 28. As illustrated, catheter portion 13 is positioned in a suitably-shaped cradle 29 formed in lower mold portion 28. Upper mold portion 26 is shaped to include voids 25, and a projecting portion 27. When the mold is closed as illustrated in the figure, heat and pressure are applied in conventional fashion to mold inwardly contoured segment 13 from the tubular catheter body 11. Following cessation of the heat and pressure, the mold and the catheter are cooled, and the upper mold portion is removed. Prior to removing the mandril from the catheter, the valve slit may then be cut through the catheter wall of the contoured portion using a knife or other straight-edge device. Those skilled in the art can readily arrive at appropriate molding conditions for a particular catheter, such as the molding time and temperature, without undue experimentation, taking into account relevant factors such as the type and composition of catheter, and the specific shape intended to result therefrom.

The formation of an effective two-way valve can be problematic. It is, or course, important that the valve be capable of opening sufficiently to enable flow of fluid in each direction through the valve, and properly re-sealing following fluid flow. The valve of the present invention is formed in the catheter in a manner that provides a very specific contour. This contour enables the valve to function effectively in both directions. For best results, the cross-sectional dimensions of the valved catheter may be optimized for the particular material from which the catheter is formed. In other words, the specific dimensions, shape, etc., of the inwardly-contoured segment that are required to form an effective two-way seal may vary from material to material. When the teachings of the present invention are utilized, no more than routine experimentation with a particular composition will be required to determine optimal parameters for that composition.

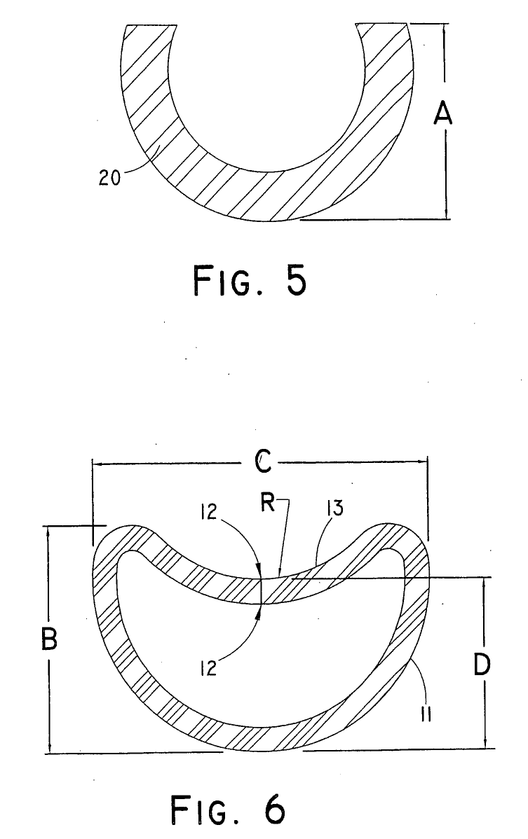

FIG. 6 illustrates an enlarged cross-sectional view of the inwardly contoured segment 13 of a polycarbonate urethane catheter body 11 following molding. FIG. 5 is an enlarged view of mandril 20 which has been removed from inwardly contoured segment 13 to arrive at the view of FIG. 6. In this embodiment, mandril 20 is fabricated from 0.062 inch (1.6 mm) outer diameter (“OD”)×0.042 inch (1.1 mm) inner diameter (“ID”) stainless steel tube. Preferably, mandril 20 has a length that exceeds the length of inwardly contoured segment 13. This additional length provides stability to the catheter adjacent to the contoured portion. In the embodiment shown, dimension “A” of mandril 20 in FIG. 5 is 0.040 inch (1 mm).

In a preferred embodiment, the catheter body 11 from which inwardly contoured segment 13 is formed has an OD of 0.100 inch (2.5 mm) and an ID of 0.062 inch (1.6 mm). As further illustrated in FIG. 6, following molding of the contoured portion, catheter body 11 has dimension “B” of 0.078 inch (2 mm), dimension “C” of 0.1 16 inch (2.9 mm), and dimension “D” of 0.052 inch (1.3 mm). Mold 24, and more particularly, mold projecting portion 27 (FIG. 4), are sized and dimensioned such that the radius “R” of contoured portion 13 in the embodiment shown is 0.031 inch (0.8 mm). By providing dimensions as described herein, the two-way valve functions very favorably in both directions when the performance material comprises a polycarbonate urethane. Thus, valve slit 12 will open when subjected to a predetermined positive or negative pressure, but will remain closed when the pressure differential across the valve is below the predetermined level. Those skilled in the art will appreciate that the dimensions provided herein are exemplary only, and that other dimensions may be appropriate for a particular performance material.

It is known in the art to form a valve by cutting a longitudinal slit through a tubular wall of a catheter. Typically, however, such valves only operate efficiently to control fluid flow in a single direction. Thus, for example, when a fluid is to be injected from a catheter into a vessel, the valve opens outwardly to permit such fluid flow when the pressure of the fluid inside the catheter exceeds the pressure on the outside of the catheter. When the fluid flows through the valve in this direction, the valve normally operates in a very efficient manner to permit the requisite fluid flow, and to re-seal once the pressure has been relieved. On the other hand, when fluid is to be aspirated from the vessel into the catheter, the higher exterior pressure tends to hold the slit closed, and the walls which configure the slit essentially form an arch. In this event, the valve may not permit efficient aspiration of the fluid, and even if so, may not re-seal properly following flow of the fluid through the valve. However, by locally reshaping the wall of the catheter to form an inwardly contoured shape as described herein, the arch effect can be overcome such that the walls which configure the valve are deflectable in an inward direction. The (un-contoured) catheter wall longitudinally adjacent to the contoured portion provides sufficient structure to minimize inward deflections of the supporting catheter wall, thereby preventing the valve from pinching tighter than desired and failing to open. The valve opening pressures are a function of the dimensions and geometry of the valve, as well as the modulus (stiffness) of the material from which the catheter is formed. In practice, the valve walls should have reasonable thickness so that even if the two sides of the slit are slightly mismatched, the slit will not leak. Those skilled in the art can readily determine an appropriate thickness without undue experimentation when following the teachings of this invention.

While a catheter material having various combinations of dimensions can provide the requisite performance characteristics, the optimal conditions for a particular catheter may be influenced by the material from which the catheter is formed. Preferably, the catheter is formed from a material that is shapeable to form an inwardly contoured segment as described. The particular material that may be utilized in a catheter may be determined utilizing no more than routine experimentation when the teachings of the present invention are followed. Thus, variables such as the modulus of the catheter material, shape and radius of the contour, thickness of the walls, etc., may be varied as desired to provide an optimal catheter and two-way valve for a particular material.

As stated above, it is preferred to form the catheter from a performance material, such as polycarbonate urethane. However, the catheter need not necessarily be formed from a performance material, as long as the material utilized is capable of forming an effective two-way valve when contoured as described herein. One example of a suitable non-performance material is silicone, a thermoset material. When a silicone catheter is utilized, the catheter is preferably formed by extrusion. However, in this case the extruded catheter will only be partially cured. This is accomplished by monitoring the time and temperature of the curing operation, and then removing the catheter before full curing has taken place. One end of the partially-cured catheter may then be re-shaped in a mold to form the inward contour as described, and the entire catheter may then be heated to arrive at the final curing.

According to the preferred embodiment described above, the inwardly contoured segment may be formed by molding a generally cylindrical tube/catheter. However, there are numerous other ways in which a catheter may be provided with an inwardly contoured segment, any such methods being within the scope of the invention. For example, a length of catheter tubing can be extruded to have one or more inwardly contoured segments, or depressions, disposed intermittently along the length of the tubing. This tubing can then be cut into individual catheters, each one of which includes an inwardly contoured segment as described. Those skilled in the art are readily able to arrive at other such methods utilizing no more than routine experimentation.

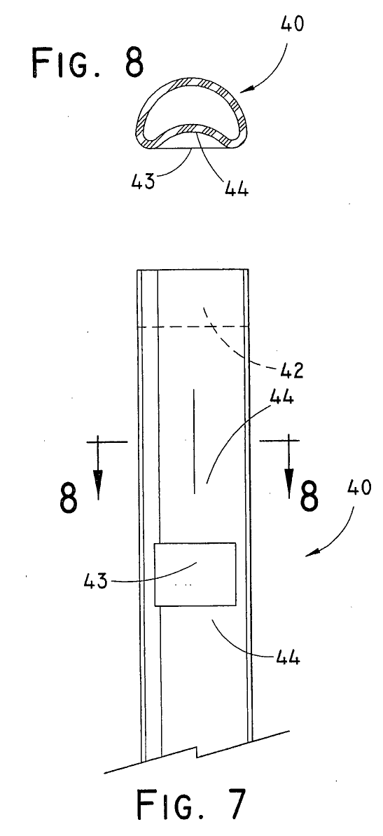

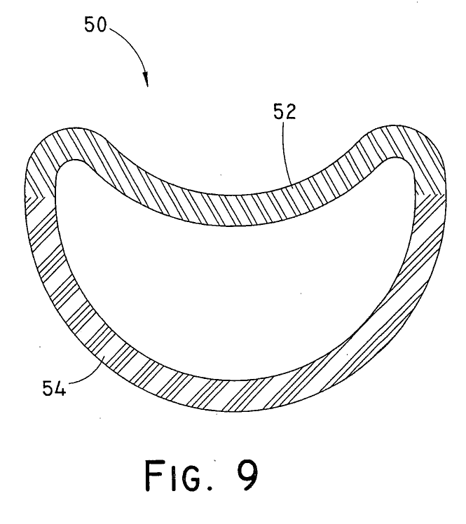

In an alternative embodiment of the present invention, a catheter 40 may be provided in which the entire length of the catheter is extruded or otherwise formed to include the cross-sectional “inwardly contoured” shape. In this embodiment, the areas 42, 43 longitudinally adjacent to the contoured portions 44 are buttressed or otherwise reinforced, as illustrated in FIGS. 7 and 8. Buttressing may be accomplished, e.g., by adding material to the buttressed portion of the catheter, thereby increasing the stiffness and/or rigidity of the catheter at the buttressed portions. Preferably, the inward contour is flattened out at the buttressed portions, as best shown in FIG. 8. Distal to the valve, buttressed portion 42 may be established by inserting a plug into the interior of the catheter. Proximal to the valve, buttressed portion 43 may be created by adding a suitable filler material. Those skilled in the art will appreciate that these are only examples of possible ways for buttressing the catheter body, and that other methods may be substituted for those specified herein.

As a still further alternative, catheter 50 may be co-extruded in a manner such that the inwardly contoured segment 52 is formed from a softer, lower durometer material, while the outer, non-contoured portion 54 is formed from a stiffer, higher durometer material. This embodiment is illustrated in FIG. 9. Preferably, the co-extrusion is carried out using materials having otherwise generally similar characteristics and properties, in order to ensure that a strong bond may be formed between the co-extruded portions. The co-extruded materials will also preferably have similar thermal expansion properties, so that the overall shape of the catheter will not change appreciably with the change in temperature. A co-extruded catheter may be formed, e.g., by co-extruding a 50A durometer silicone with an 80A durometer silicone. Similarly, a catheter may be formed by co-extruding two polyurethanes of different durometers. Those skilled in the art will appreciate that numerous other combinations of materials for inwardly contoured segment 52 and non-contoured portion 54 may be co-extruded in this manner to form a suitable catheter.

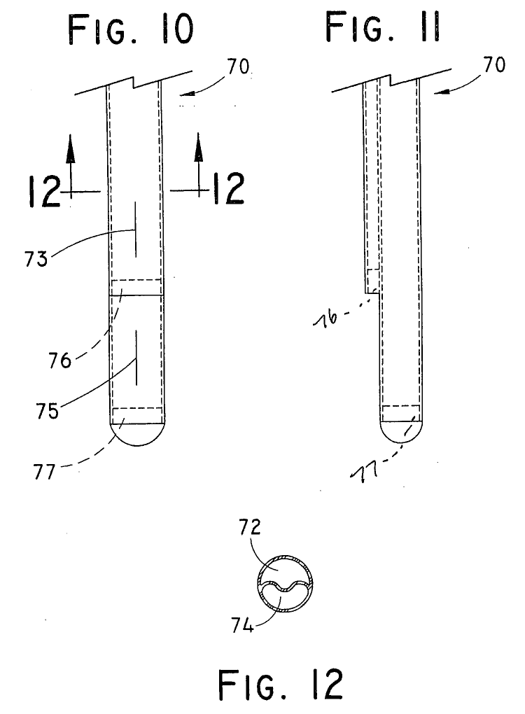

Another alternative embodiment of a valved catheter 70 is illustrated in FIGS. 10-12. Catheter 70 is a dual-lumen valved catheter, wherein lumen 72 has a one-way valve 73, and lumen 74 has a two-way valve 75. In the embodiment shown, the catheter wall at each longitudinal end of two-way valve 75 is buttressed or otherwise supported against inward deflections. At one longitudinal end, the valve is supported by the complete circle of the catheter, which in turn, is supported by a plug 76 positioned inside catheter 70. At the other longitudinal end, the valve is supported by the buttressed tip 77. As still further variations, the catheter can be provided with any combination of the alternative features described above. Thus, for example, the number and shape of lumens may be varied as desired. Similarly, the number of valves of the catheter may be varied, and any desired combination of one-way and two-way valves may be provided.

It is therefore intended that the foregoing detailed description be regarded as illustrative rather than limiting, and that it be understood that it is the following claims, including all equivalents, that are intended to define the spirit and scope of this invention.

1. A catheter for use in fluid transport between a body vessel of a patient and a site external of the patient, comprising:
a catheter body sized to at least partially extend between said body vessel and said external site, said catheter body having a proximal portion extending toward said external site and a distal portion extending to said body vessel, said catheter body comprising an inwardly contoured segment at said distal portion; and
a valve formed at said inwardly contoured segment, said valve operable to open in a first direction when subjected to a first pressure from a side of the valve to permit fluid flow therethrough in said first direction, said valve operable to open in a second direction, opposite to said first direction, when subjected to a second pressure from another side of the valve to permit fluid flow therethrough in said second direction, said valve structured to remain substantially closed in the absence of said first and second pressures.

2. The catheter of claim 1, wherein said valve comprises a longitudinal slit formed at said contour.

3. The catheter of claim 1, wherein said catheter is formed of a performance material.

4. The catheter of claim 1, wherein said catheter is formed of a thermoplastic polymer.

5. The catheter of claim 3, wherein said catheter is formed of polycarbonate urethane.

6. The catheter of claim 2, wherein a longitudinal portion of said catheter axially adjacent to said inwardly contoured segment is buttressed.

7. The catheter of claim 2, wherein a longitudinal portion of said catheter beyond each axial end of said inwardly contoured segment is buttressed.

8. The catheter of claim 2, wherein said catheter comprises a co-extrusion, said co-extrusion comprising a relatively stiff outer catheter portion, and comprising a relatively soft inwardly contoured segment.

9. The catheter of claim 2, wherein said catheter comprises two lumens, and wherein said valve provides communication between a first one of said lumens and a space exterior of said catheter, further comprising a second valve for providing communication between a second one of said lumens and a space exterior of said catheter.

10. The catheter of claim 9, wherein said second valve comprises a one-way valve.

11. A method of forming a two-way valved catheter suitable for use in transporting a fluid between a body vessel of a patient and a site external of the patient, comprising:
providing a tubular catheter body, said catheter body sized such that a proximal portion extends substantially to said external site and a distal portion extends to said body vessel;
forming an inwardly contoured segment at said distal portion of said tubular catheter body; and
forming a valve at said inwardly contoured segment, said valve comprising a longitudinal slit formed in said segment, said valve being sized and dimensioned such that upon exposure to a first predetermined pressure from a first side thereof, said valve opens in a first direction to permit fluid flow therethrough in said first direction, and upon exposure to a second predetermined pressure from a second side thereof, said valve opens in a second direction, opposite to said first direction, to permit fluid flow therethrough in said second direction, said valve structured to remain closed in the absence of said predetermined first and second pressures.

12. The method of claim 11, wherein said valve is formed by:
positioning a distal portion of said catheter in a mold;
molding said distal portion to form said inwardly contoured segment; and
cutting said slit in said inwardly contoured segment.

13. The method of claim 12, wherein said molding step comprises:
positioning a mandril in an interior space of said distal portion, said mandril sized and shaped for forming said inwardly contoured segment; and
molding said segment with said mandril positioned therein.

14. The method of claim 11, wherein said tubular catheter body is formed by extrusion.

15. The method of claim 14, wherein the extruded tubular catheter body is partially cured, and said inwardly contoured segment is formed in said partially cured catheter body, and wherein said catheter body having said inwardly contoured segment formed therein is substantially fully cured.

16. The method of claim 11, wherein said catheter body is formed from a performance material.

17. The method of claim 11, wherein said catheter body is formed from a polycarbonate urethane.

18. The method of claim 11, wherein said tubular catheter body comprises a co-extrusion, and wherein said inwardly contoured segment is relatively soft, and a non-contoured outer portion is relatively stiff.

19. The method of claim 11, wherein said tubular catheter body comprises two lumens, and wherein said valve provides communication between one of said lumens and a space exterior of said catheter, and wherein a second valve is formed in said catheter body for providing communication between the other lumen and a space exterior of said catheter.

20. The method of claim 19, wherein said second valve comprises a one-way valve.
